(19) United States
(12) Patent Application Publication
(10) Pub. No.: US 2026/0308105 A1
(43) Pub. Date: Oct. 8, 2026

(54) A PROCESS FOR THE PREPARATION OF CRYSTALLINE FORM A OF N, N'-BIS(2-AMINOETHYL)-1,2-ETHANEDIAMINE TETRAHYDROCHLORIDE

(71) Applicant: BIOPHORE INDIA PHARMACEUTICALS PVT. LTD, Telangana Hyderabad (IN)

(72) Inventors: Manik Reddy PULLAGURLA, Telangana Hyderabad (IN); Kiran Kumar KOTHAKONDA, Telangana Hyderabad (IN); Bhaskar Reddy PITTA, Telangana Hyderabad (IN); Rajesham BOGE, Telangana Hyderabad (IN); Jagadeesh Babu RANGISETTY, Telangana Hyderabad (IN)

(21) Appl. No.: 18/996,266

(22) PCT Filed: Jul. 19, 2023
(86) PCT No.: PCT/IB2023/057358

§ 371 Date: Jan. 17, 2025

(30) Foreign Application Priority Data
Jul. 19, 2022 (IN) 202241041141

(51) Int. Cl. C07C 209/62; C07C 209/84

(52) U.S. Cl. CPC C07C 209/62; C07C 209/84; C07B 2200/13

(57) ABSTRACT

The present invention relates to a process for the preparation of crystalline form A of N,N′-bis(2-aminoethyl)-1,2-ethanediamine tetrahydrochloride (I).

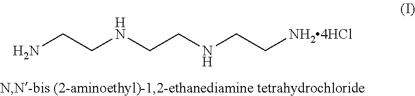

### FIELD OF THE INVENTION

The present invention provides a process for the preparation of crystalline form A of N,N′-bis(2-aminoethyl)-1,2-ethanediamine tetrahydrochloride (I).

### BACKGROUND OF INVENTION

Trientine tetrahydrochloride (I) is N,N′-bis(2-aminoethyl)-1,2-ethanediamine tetrahydrochloride. It is marketed in US as Cuvrior® (300 mg) tablet for oral administration. Cuvrior® is a copper chelator indicated for the treatment of adult patients with stable Wilson's disease who are de-coppered and tolerant to penicillamine.

The synthesis of Trientine tetrahydrochloride was reported in many patents and non-patent literature. The contents of which are hereby incorporated as reference in their entirety.

EP 1 778 618 describes synthetic techniques for producing triethylenetetramine and its salts including the 0.2 HCl salt and the 0.4 HCl salt. Only the 2 HCl salt is said to be useful in the treatment of Wilson's disease.

WO 2006/027705 describes the synthesis of triethylenetetramines, including Form I and Form II triethylenetetramine dihydrochloride. This document does not mention the crystalline forms of triethylenetetramine tetrahydrochloride.

U.S. Pat. No. 10,988,436 claim crystalline form B and discloses crystalline form A of Trientine tetrahydrochloride and process for the preparation thereof.

### OBJECTIVE OF THE INVENTION

The main objective of the present invention provides a process for the preparation of crystalline form A of Trientine tetrahydrochloride (I).

Another objective of the present invention provides crystalline form A of Trientine Tetrahydrochloride (I) with high yield.

### SUMMARY OF THE INVENTION

Accordingly, in one aspect of the present invention is to provide a process for the preparation of crystalline form A of Trientine Tetrahydrochloride (1) as shown in scheme 1, the process comprising:
a) adding of 2-chloroacetonitrile (VI) to ethane-1,2-diamine (V) in presence of suitable base to form 2,2′-(ethane-1,2-diylbis(azanediyl)) diacetonitrile (IV);
b) protecting the free amino groups using suitable protecting groups to obtain the Boc protected Di-tert-butyl ethane-1,2-diylbis(cyanomethyl carbamate) (III);
c) reducing intermediate (III) by hydrogenation using Raney nickel in the presence of methanolic ammonia and converting to difumarate salt by using fumaric acid to yield Boc protected Di-tert-butyl ethane-1,2-diylbis(2-aminoethylcarbamate) difumarate (II);
d) deprotecting intermediate (II) by treating with concentrated hydrochloric acid to yield Trientine tetrahydrochloride crude (Ia); and
e) purifying crude Trientine tetrahydrochloride (Ia) to obtain crystalline form A of Trientine Tetrahydrochloride (I), wherein the purification process comprises:
   1. dissolving crude Trientine tetrahydrochloride (Ia) in a protic solvent,
   2. heating the reaction mass of step 1), optionally adding anti-solvent, and
   3. isolating crystalline form A of Trientine tetrahydrochloride.

In another aspect of the present invention is to provide crystalline form A of Trientine tetrahydrochloride (I) with high yield.

### BRIEF DESCRIPTION OF THE DRAWINGS

FIG. 1 illustrates X-Ray Diffraction (XRD) pattern of crystalline form A of Trientine tetrahydrochloride (I)

FIG. 2 illustrates Differential Scanning calorimetry of crystalline form A of Trientine tetrahydrochloride (I)

FIG. 3 illustrates Therapeutic Goods Administration of crystalline form A of Trientine tetrahydrochloride (I)

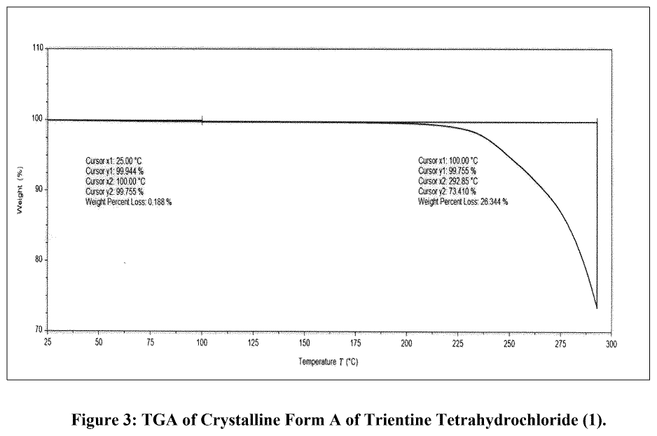

FIG. 4 illustrates Raman spectra of crystalline form A of Trientine tetrahydrochloride (I)

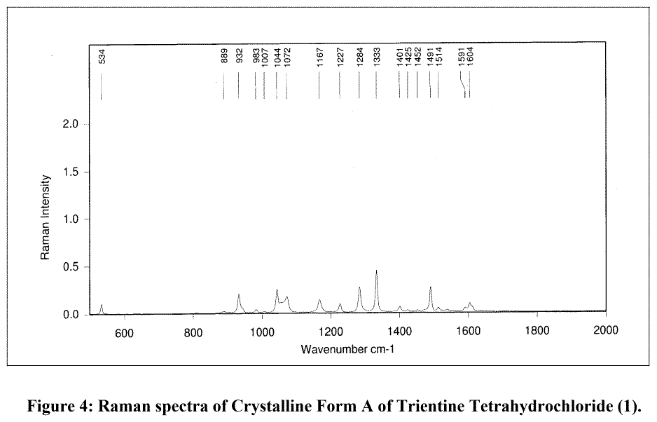

### DETAILED DESCRIPTION OF THE INVENTION

Accordingly, in one embodiment of the present invention is to provide a process for the preparation of crystalline form A of Trientine tetrahydrochloride (1) as shown in scheme 1

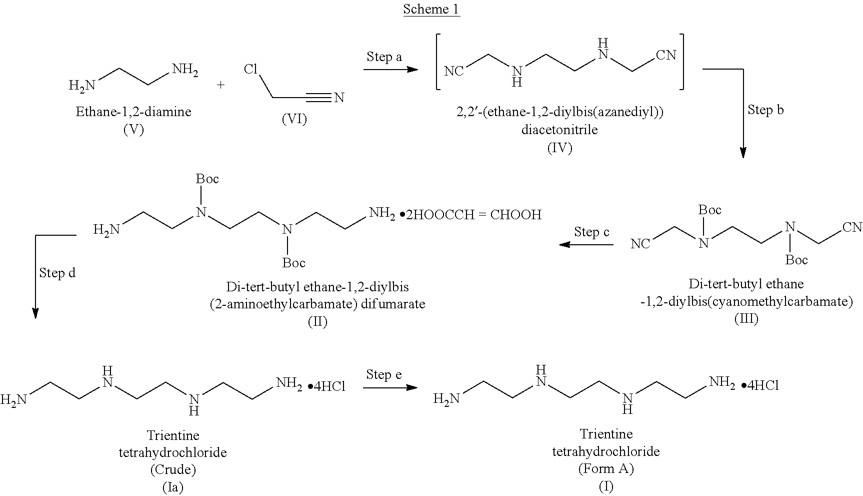

In another embodiment, the steps involved in the preparation of Trientine Tetrahydrochloride (1) as shown in scheme 1 are as follows:

In some embodiment, step a) of the present invention involves the addition of 2-chloroacetonitrile (VI) to ethane-1,2-diamine (V) in the presence of a base in a suitable aprotic solvent at 25-30° C. to obtain 2,2′-(ethane-1,2-diylbis(azanediyl) diacetonitrile (IV),

In another embodiment, step b) of the present invention proceeds by protecting the amino groups of intermediate (IV) using suitable protecting groups. The reaction filtrate obtained in step a) containing intermediate (IV) was cooled and reacted with a suitable protecting group. On completion of reaction, the final residue was washed with different volumes of a suitable aprotic solvent and dried under vacuum to obtain Di-tert-butyl ethane-1,2-diyl bis(cyanomethyl carbamate) (III). The two free amine groups of intermediate (IV) were protected by treating with t-butyl carbamate (Boc) anhydride to yield Di-tert-butyl ethane-1,2-diylbis(cyanomethyl carbamate) (III), which is further purified by preparing slurry in cyclohexane or ethyl diisopropyl ether.

The suitable amino protecting groups were selected from a group comprising of Fluorenyl methyloxy carbonyl (Fmoc), tert-butyloxycarbonyl (BOC), phthalimide, toluene sulfonyl (Ts), methane sulfonyl (Ms), triphenylmethyl (Trityl), carboxy benzyl (CBZ) or the like, tert-butyloxycarbonyl (Boc) was used in the present invention.

In another embodiment, step c) of the present invention proceeds with the reduction of the cyano group of intermediate (III) with methanolic ammonia and catalyst, preferably Raney-nickel was used as a catalyst. On completion of reaction, the residue so obtained was treated with fumaric acid dissolved in a protic solvent and heated at 50-70° C. The reaction mixture was cooled and the solid so formed was washed with a mixture of protic solvents and an aprotic solvent to obtain Di-tert-butyl ethane-1,2-diylbis(2-aminoethylcarbamate) difumarate (II).

In another embodiment, step d) involves deprotection of amino group from intermediate (II), and conversion of fumarate salt to Trientine tetrahydrochloride crude (Ia), which was reacted with concentrated hydrochloric acid and heated at 50-70° C. On completion of reaction, reaction mass was cooled. The conversion of difumarate salt (II) to Trientine tetrahydrochloride crude (Ia) as described above is not reported in any prior art, which improves the purity of intermediate (II).

In another embodiment, step e) involves purifying Trientine tetrahydrochloride crude (Ia) to obtain crystalline form A of Trientine Tetrahydrochloride (I), wherein the purification process comprises:
1. dissolving crude Trientine tetrahydrochloride (Ia) in a protic solvent;
2. heating the reaction mass of step 1), optionally adding anti-solvent, and
3. isolating crystalline form A of Trientine tetrahydrochloride.

The suitable bases used in step a) and step c) were selected from potassium carbonate, sodium carbonate, potassium hydroxide, sodium hydroxide, sodium bicarbonate, potassium bicarbonate, triethylamine, pyridine, aqueous ammonia, methanolic ammonia, group of metal alkoxides comprising of sodium methoxide, sodium ethoxide; potassium methoxide, potassium ethoxide, sodium butoxide, potassium butoxide, potassium-t-butoxide, sodium tertiary butoxide or the like. Preferably potassium carbonate, methanolic ammonia, sodium hydroxide, sodium carbonate, sodium methoxide and the like.

In another embodiment, step a), step b), step c), and step d) carried out in a solvent selected from aprotic or protic solvent or mixtures thereof.

The suitable aprotic solvent used in step a), step b), step c), and step d) were selected from acetone, acetonitrile, 1,4-dioxane, diethyl ether, dichloromethane, ethyl acetate, N, N-dimethylformamide, methyl tertiary butyl ether, hexane, cyclohexane, toluene, tetrahydrofuran or the like, preferably acetone, acetonitrile, dichloromethane, ethyl acetate, cyclohexane, or mixtures thereof.

The suitable protic solvent used in step b), step c), step d) and step e) were selected from a group comprising of water, methanol, ethanol, isopropyl alcohol (IPA), n-propanol, n-butanol, or the like, preferably water, methanol, ethanol, isopropyl alcohol, or mixtures thereof.

The suitable anti-solvent used in step e) is selected from ethanol, methanol, isopropanol, acetone, or mixtures thereof.

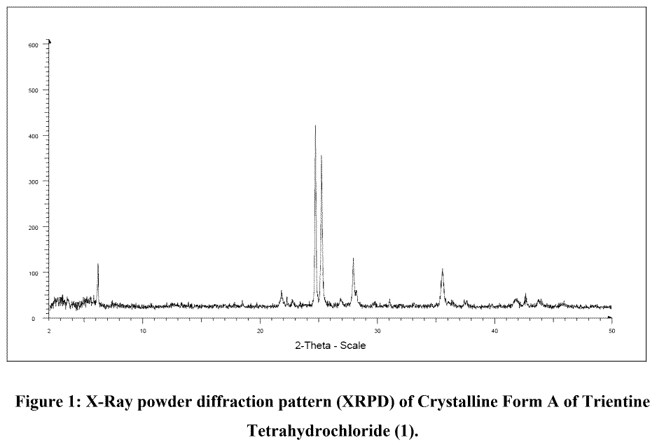

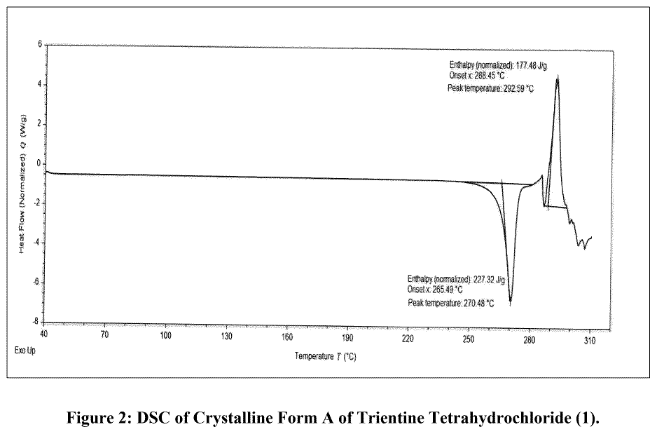

In another embodiment, crystalline form A of Trientine tetrahydrochloride (1) obtained according to the present invention is characterized by X-ray powder diffractions (XRD) pattern as shown in FIGS. 1 and 2 theta values as provided in Table 1:

TABLE 1

| Two theta (2Θ) values | Relative Intensity I/Io (%) |
|---|---|
| 6.12 | 28.2 |
| 21.82 | 14.1 |
| 22.28 | 10.5 |
| 22.76 | 8.6 |
| 24.70 | 100 |
| 25.22 | 84.4 |
| 26.87 | 9.4 |
| 27.95 | 30.9 |
| 28.22 | 13.6 |
| 31.04 | 9.5 |
| 35.55 | 25.5 |
| 41.77 | 9 |
| 42.68 | 12.4 |
| 43.74 | 8.6 |

In another embodiment, the crystalline form A of Trientine tetrahydrochloride (1) has a melting temperature of about 270° C. as measured by Differential Scanning calorimetry.

The following examples further illustrate the present invention but should not be construed in any way as to limit its scope.

### EXAMPLES

### Example 1: Preparation of 2,2′-(Ethane-1,2-diylbis(azanediyl))diacetonitrile (IV)

100 g (1.663 moles) of ethane-1,2-diamine (V) was dissolved in 1200 mL of acetonitrile at 25-30° C., to this 460 g (3.326 moles) of potassium carbonate was added. The reaction mass was cooled to 15-20° C. and 276.8 g (3.658 moles) of 2-chloroacetonitrile (VI) was added to the reaction mass over a period of 40-45 min at 15-20° C. The temperature of the reaction mass was raised to 25-30° C. and stirred for 22-24 hrs. On completion of the reaction, the reaction mass was filtered at 25-30° C. and the solid washed with 200 mL of acetonitrile. The filtrate so obtained was cooled to 15-20° C. to obtain 2,2′-(Ethane-1,2-diylbis(azanediyl)) diacetonitrile (IV). The obtained filtrate was used in the next step without isolation.

### Example 2: Preparation of Di-tert-butyl ethane-1,2-diylbis(cyanomethyl carbamate) (III)

1088 g (4.99 moles) of tertiary-butyl carbamate (Boc) anhydride was added to the filtrate containing 2,2′-(ethane-1,2-diylbis(azanediyl)) diacetonitrile (IV) obtained in the example−1 over a period of 40-60 min at 15-20° C. The reaction mass was maintained for 12-14 hrs. On completion of the reaction, the solvent was distilled off under vacuum below 55° C. and the residue was cooled to 25-30° C. 2000 mL of ethyl acetate was added to the residue and stirred for 15-30 min at 25-30° C. 10% aqueous sodium chloride solution was added to the reaction mass at 25-30° C., stirred for 15-30 min and then allowed stand for 15-30 min. Again, same procedure was followed with 10% of sodium chloride solution. The organic layer was distilled off completely and 1000 mL of cyclohexane was added to the residue. The reaction mass was stirred and the solid so formed was filtered. Finally, the wet cake so obtained was washed with 200 mL of cyclohexane at 25-30° C. Again, the wet cake was charged with 1500 mL of cyclohexane in a clean and dry RB flask, stirred at 25-30° C. for 60-90 min. The solid obtained was filtered and washed with 200 mL of cyclohexane followed by suck dried under vacuum below 40° C. to obtain pure tert-butyl ethane-1,2-diylbis(cyanomethyl carbamate) (III). Yield: 71%; Purity by Gas chromatography (GC): 99.0%.

### Example 3: Preparation of Di-tert-butyl ethane-1,2-diylbis(2-aminoethylcarbamate) difumarate (II)

100 g of tert-butyl ethane 1,2diylbis(cyanomethyl carbamate) (III) was charged in a clean and dry autoclave and 1000 mL of methanolic ammonia was added to it at 25-30° C. 50 g of Raney nickel (rinsed with purified water and methanol) was added to the reaction mass at 25-30° C., under nitrogen atmosphere. A pressure of 2-3 Kg of nitrogen gas was applied to the autoclave and the reaction mass stirred for 5-10 min at 25-30° C. The nitrogen gas was released, and again repeated the same procedure. Then, the reaction mass was maintained under hydrogen gas at a pressure of 2-3 Kg for 24-28 hrs. After completion of the reaction, the reaction mass allowed to settle for 20-30 min at 25-30° C. and filtered through Hyflo. The filtrate was distilled under vacuum and 200 mL of isopropyl alcohol was added to the residue. The solvent was distilled under vacuum below 45° C. and the residue obtained was cooled to 25-30° C. 700 mL of isopropyl alcohol and 100 g of fumaric acid dissolved in 300 mL of methanol were added to the residue at 25-30° C. The reaction mass was heated for 45-60 min at 60-65° C. and then cooled to 25-30° C. It was further cooled to 0-5° C. for 5-6 hrs. The precipitated solid was filtered and washed with chilled isopropyl alcohol:methanol solvent mixture (7:3) and 200 mL of chilled acetone and dried under vacuum. The solid so obtained was further dissolved in a mixture of (7:3) isopropyl alcohol:methanol at 25-30° C., heated for 60-90 min at 65-70° C. The reaction mass was then cooled to 25-30° C. and stirred for 90-120 min. Further the reaction mass was cooled to 0-5° C. for 3-4 hrs. The solid so formed was filtered, washed with chilled acetone, and dried under vacuum below 40° C. to obtain di-tert-butyl ethane-1,2-diylbis(2-aminoethylcarbamate) difumarate (II) salt. Yield: 35%; Purity by HPLC: 99.5%

### Example 4: Preparation of Trientine Tetrahydrochloride Crude (Ia)

To 100 g (0.172 moles) of di-tert-butyl ethane-1,2-diylbis(2-aminoethylcarbamate) difumarate salt (II), 300 mL of concentrated hydrochloric acid was added at 25-30° C. The reaction mass was heated at 65-70° C. for 1-2 hrs, then cooled to 25-30° C. and maintained for 8-10 hrs to obtain Trientine Tetrahydrochloride crude (Ia).

### Example 5: Preparation of Crystalline form A of Trientine Tetrahydrochloride (I)

50 g of Trientine tetrahydrochloride crude (Ia) and 75 ml of water was taken at 25 to 30° C. The reaction mass was heated to 60-65° C. and stirred for 15 to 30 minutes. To this 5.0 g of activated carbon was added and stirred. Filtered the mass and washed with 25 ml of water to get the filtrate. The obtained filtrate was heated to 60-65° C. To this 500 ml of hot methanol was added and stirred. Cooled the reaction mass to 30 to 35° C. and stirred. Filtered the solid and washed with water. Dried the material to get the titled compound. Yield: 85%.

### Example 6: Preparation of Crystalline Form A of Trientine Tetrahydrochloride (I)

30 g of Trientine tetrahydrochloride crude (Ia) and 30 ml of water was taken at 25 to 30° C. The reaction mass was heated at 75 to 80° C. and stirred for 15 to 30 minutes. To this 300 ml of hot Ethanol was added and stirred. Cooled the reaction mass to 30 to 35° C. and stirred. Filtered the solid and washed with 60 ml of ethanol. Dried the material to get the titled compound. Yield: 86.6%

### Example 7: Preparation of Crystalline Form A of Trientine Tetrahydrochloride (I)

30 g of Trientine tetrahydrochloride crude (Ia) and 100 ml of methanol was taken at 25 to 30° C. The reaction mass was heated at 60 to 65° C. and stirred for 60 to 90 minutes. Cooled the reaction mass to 30 to 35° C. and stirred. Filtered the solid and washed with 20 ml of methanol. Dried the material to get the titled compound. Yield: 91.0%

### Example 8: Preparation of Crystalline Form A of Trientine Tetrahydrochloride (I)

5.0 g of Trientine tetrahydrochloride crude (Ia) and 7.5 ml of water was taken at 25 to 30° C. The reaction mass was heated at 50 to 55° C. and stirred for 15 to 30 minutes. To this 60 ml of hot acetone was added and stirred. Cooled the reaction mass to 30 to 35° C. and stirred. Filtered the solid and washed with 10 ml of acetone. Dried the material to get the titled compound. Yield: 96.0%

### Example 9: Preparation of Crystalline Form A of Trientine Tetrahydrochloride (I)

10 g of Trientine tetrahydrochloride crude (Ia) and 15 ml of water was taken at 25 to 30° C. The reaction mass was heated at 55 to 60° C. and stirred for 15 to 30 minutes. To this 100 ml of hot isopropanol was added and stirred. Cooled the reaction mass to 30 to 35° C. and stirred. Filtered the solid and washed with 20 ml of isopropanol. Dried the material to get the titled compound. Yield: 80%

### Example 10: Preparation of Crystalline Form A of Trientine Tetrahydrochloride (I)

5.0 g of Trientine tetrahydrochloride crude (Ia) and 10 ml of water was taken at 25 to 30° C. The reaction mass was heated at 55 to 60° C. and stirred for 15 to 30 minutes. To this 100 ml of hot isopropanol and methanol (1:1) was added and stirred. Cooled the reaction mass to 30 to 35° C. and stirred. Filtered the solid and washed with 20 ml of isopropanol and methanol (1:1). Dried the material to get the titled compound. Yield: 80%

We claim:

1. A process for the preparation of crystalline form A of Trientine Tetrahydrochloride (1), 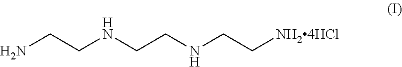
wherein the process comprising:
a) adding of 2-chloroacetonitrile (VI) 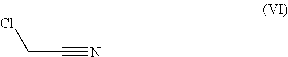
to ethane-1,2-diamine (V) 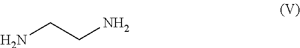
in the presence of a base to form 2,2′-(ethane-1,2-diylbis(azanediyl)) diacetonitrile (IV); 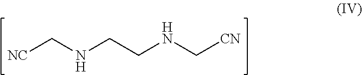
b) protecting the free amino groups using suitable protecting groups to obtain the Boc protected Di-tert-butyl ethane-1,2-diylbis(cyanomethyl carbamate) (III); 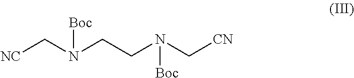 [SMILES: CC(C)(C)OC(=O)N(CC#N)CCN(CC#N)C(=O)OC(C)(C)C]
c) reducing intermediate (III) by hydrogenation using Raney nickel in the presence of methanolic ammonia and converting to difumarate salt by using fumaric acid to yield Boc protected Di-tert-butyl ethane-1,2-diylbis(2-aminoethylcarbamate) difumarate (II); 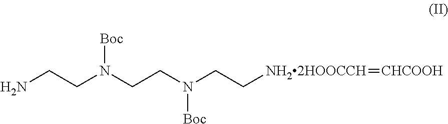
d) deprotecting intermediate (II) by treating with concentrated hydrochloric acid to yield Trientine tetrahydrochloride crude (Ia); and 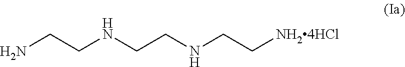
e) purifying crude Trientine tetrahydrochloride (Ia) to obtain crystalline form A of Trientine Tetrahydrochloride (I), wherein the purification process comprises:
1. dissolving crude Trientine tetrahydrochloride (Ia) in a protic solvent,
2. heating the reaction mass of step 1), optionally adding anti-solvent, and
3. isolating crystalline form A of Trientine tetrahydrochloride.

2. The process as claimed in claim 1, wherein the base used in step (a) is selected from potassium carbonate or sodium carbonate.

3. The process as claimed in claim 1, wherein the anti-solvent used in step e) is selected from ethanol, methanol, isopropanol, acetone, or mixtures thereof.

4. The process as claimed in claim 1, wherein step a), step b), step c), and step d) of carried out in a solvent selected from aprotic or protic solvent or mixtures thereof.

5. The process as claimed in claim 1, wherein the aprotic solvent used in step a), step b), step c), and step d) were selected from acetone, acetonitrile, 1,4-dioxane, diethyl ether, dichloromethane, ethyl acetate, N, N-dimethylformamide, methyl tertiary butyl ether, hexane, cyclohexane, toluene, tetrahydrofuran or the like, preferably acetone, acetonitrile, dichloromethane, ethyl acetate, cyclohexane or mixtures thereof.

6. The process as claimed in claim 1, wherein the protic solvent used in step b), step c), step d) and step e) were selected from a group comprising of water, methanol, ethanol, isopropyl alcohol (IPA), n-propanol, n-butanol or the like, preferably water, methanol, ethanol, isopropyl alcohol or mixtures thereof.
